(19) United States
(12) Patent Application Publication
(10) Pub. No.: US 2009/0112002 A1
(43) Pub. Date: Apr. 30, 2009

(54) PROCESS FOR PREPARATION OF ALDONIC ACIDS AND DERIVATIVES THEREOF

(76) Inventors: Alexander Charles Weymouth-Wilson, Cholsey (GB); Robert Clarkson, Cholsey (GB)

Correspondence Address: EVAN LAW GROUP LLC, 600 WEST JACKSON BLVD., SUITE 625, CHICAGO, IL, 60661, US

(21) Appl. No.: 11/932,961

(22) Filed: Oct. 31, 2007

(51) Int. Cl. C07D 519/00

(52) U.S. Cl. 549/305

(57) ABSTRACT

A process for the preparation of L-gluconic acid or a salt thereof, comprises treating an aqueous solution of 6-bromo-6-deoxy-2,3-anhydro-D-manno-1,4-lactone with a base at a pH of at least 12 and at a temperature of 45 to 55° C. to obtain an aqueous solution of L-gluconic acid.

### BACKGROUND

Naturally occurring glucose exists as the D-isomer and this is the isomer of choice for most applications as it is the biologically active isomer. However, in some cases, the biological inactivity of the L-isomer is useful. For instance, L-glucose can be used as a laxative or a bowel cleansing product which may be useful, for example, if a scan of the colon or rectum is required.

However, because it does not occur widely in nature, it has proved both difficult and expensive to synthesise L-glucose and its analogues. Previous processes for the synthesis of L-glucose have generally used L-arabinose as a starting material. L-arabinose is a naturally occurring sugar which is available in significant quantities from sugar beet pulp by the method described in Chemical Abstracts: 142135v, Vol. 75, 1971. According to this method, dry sugar beet pulp is treated with sulfuric acid to obtain an extract solution which is subsequently fermented, evaporated and filtered. L-arabinose is thereafter crystallized from the resulting filtrate.

L-glucose can be produced from L-arabinose by the method of Sowden and Fischer, J.A.C.S., Vol. 69 (1947), pp. 1963-1965. In accordance with this method, L-arabinose is condensed with nitromethane in the presence of sodium methoxide to provide sodium salts of the nitroalcohols. The sodium salts are readily converted to the corresponding sugars by means of the Nef reaction.

Lundt et al. (I. Lundt, C. Pedersen, Synthesis, 7, 669-672, (1992)) teach that 6-bromo-6-deoxy-2,3-anhydro-D-manno-1,4-lactone can be produced by the reaction of 2,6-dibromo-2,6-dideoxy-D-mannono-1,4-lactone with potassium fluoride under strictly anhydrous conditions. The reaction described by Lundt et al. is carried out using anhydrous potassium fluoride in anhydrous acetone and the importance of the anhydrous conditions is repeatedly emphasised.

A process for the conversion of D-glucono-1,5-lactone or a salt thereof to 2,6-dibromo-2,6-dideoxy-D-mannono-1,4-lactone is described by Lundt et al. (I. Lundt, C. Pedersen, Synthesis, 7, 669-672, (1992)). In this process the gluconolactone starting material is stirred with glacial hydrogen bromide at room temperature for 18 hours, the reaction mixture is cooled and quenched with methanol, then, after standing overnight, the reaction mixture is concentrated to a syrup, co-evaporated with methanol and then water. Following this, water is added and the product is extracted with ether.

### SUMMARY

The present invention relates to a process for the synthesis of L-gluconic acid which is higher yielding and can be carried out at lower cost than traditional methods. In particular, the method relates to a process for the conversion of 6-bromo-6-deoxy-2,3-anhydro-D-manno-1,4-lactone to L-gluconic acid. Furthermore, the process optionally includes further steps for the production of the starting material, 6-bromo-6-deoxy-2,3-anhydro-D-manno-1,4-lactone, from the readily available compound D-glucono-1,5-lactone. Additionally, it includes optional steps for the conversion of L-gluconic acid to L-glucose and analogues of L-glucose. The present invention can be extended to the preparation of epoxides from bromohydrins.

### DEFINITIONS

A bromohydrin is an organic compound containing a bromine and a hydroxyl on adjacent carbons.

An epoxide is an organic compound with a three-member ring containing two carbons and an oxygen. A chemical reaction which forms an epoxide is an epoxidation.

A lactone is an organic compound with a ring containing an —O—C(O)— moiety.

A α-bromohydrin lactone is an organic compound that is both a bromohyrdin and a lactone, and the bromine of the bromohydrin is on the carbon adjacent the carbonyl (i.e. the C(O) moiety) of the lactone.

An aldonic acid is a compound of the formula HOOC—(CHOH)ₙ—CH₂OH, where n is 1 to 7. Preferably n is 3 or 4. Preferably, the aldonic acid is L- or D-gluconic acid.

An aldonolactone is a lactone of an aldonic acid, preferably containing 3 to 9 carbons, more preferably 5 or 6 carbons.

An α-bromohydrin aldonolactone is an organic compound that is both a bromohyrdin and an aldonolactone, and the bromine of the bromohydrin is on the carbon adjacent the carbonyl (i.e. the C(O) moiety) of the lactone. Preferably, the α-bromohydrin aldonolactone contains 3 to 9 carbons, more preferably 5 or 6 carbons.

An epoxyaldonolactone is an aldonolactone which is an epoxide. A α-epoxyaldonolactone is an epoxyaldonolactone in which the oxygen of the epoxide is on the carbon adjacent the carbonyl (i.e. the C(O) moiety) of the lactone. Preferably, the epoxyaldonolactone contains 3 to 9 carbons, more preferably 5 or 6 carbons.

An organic solvent is a solvent containing carbon.

A monosaccharide is a molecule with the chemical formula (CH₂O)ₙ₊ₘ with the chemical structure H(CHOH)ₙC═O(CHOH)ₘH, where m and n are integers and m+n is at least two. If either n or m is zero, the monosaccharide comprises an aldehyde group and is termed an aldose; otherwise it comprises a ketone group and is termed a ketose. At least one-half of the non-carbonyl carbon atoms of the monosaccharide have a hydroxyl substituent. Example monosaccharides include aldotetroses such as erythrose and threose; ketotetrose such as erythrulose; aldopentoses such as arabinose, lyxose, ribose and xylose; ketopentoses such as ribulose and xylulose; aldohexoses such as allose, altrose, galactose, glucose, gulose, idose, mannose and talose; ketohexoses such as fructose, psicose, sorbose and tagatose; keto-heptoses such as mannoheptulose and sedoheptulose; octoses such as octolose and 2-keto-3-deoxy-manno-octonate; nonoses such as sialose.

An oligosaccharide is a polymer containing two to ten component monosaccharides. Example oligosaccharides include sucrose, lactose, maltose, trehalose and cellobiose.

A polysaccharide is a saccharide polymer containing more than ten component monosaccharides. Example polysaccharides include starch, cellulose and dextran.

A saccharide is a monosaccharide, an oligosaccharide or a polysaccharide, and saccharides with one more substituents, where the substituents may be, for example, halide, amine, C₁-C₅ alkyl, aminoacid, protein, nucleoside, nucleotide, phosphate, sulphate and carboxy.

### DETAILED DESCRIPTION

The present invention is based on the discovery of a new process for the preparation of L-gluconic acid from D-glucono-1,5-lactone, which includes three different aspects of the present invention: the preparation of 2,6-dibromo-2,6-dideoxy-D-mannono-1,4-lactone from D-glucono-1,5-lactone (third aspect of the present invention); the preparation of 6-bromo-6-deoxy-2,3-anhydro-D-manno-1,4-lactone from 2,6-dibromo-2,6-dideoxy-D-mannono-1,4-lactone (second aspect of the present invention); and the preparation of L-gluconic acid from 6-bromo-6-deoxy-2,3-anhydro-D-manno-1,4-lactone (first aspect of the present invention). This process can be further extended by converting the L-gluconic acid to L-gluconolactone, and then converting the L-gluconolactone to L-glucose.

The second aspect of the present invention can be extended to the preparation of epoxides from bromohydrins. In particular, the second aspect of the present invention takes advantage of the discovery that the preparation of epoxides from bromohydrins proceeds particularly well if a catalytic amount of water is present in the reaction mixture. Shorter reaction times and higher yields are achieved as compared to the strictly anhydrous conditions previously used to carry out the reaction, thereby obtaining superior results, without expensive anhydrous solvents.

The preparation of L-gluconic acid from 6-bromo-6-deoxy-2,3-anhydro-D-manno-1,4-lactone is known (I. Lundt, R. Madsen, Top. Curr. Chem., 215, 177-191, (2001)) but has always previously been conducted by the ice cold addition of the base to the starting material followed by allowing the reaction to proceed for three days. In contrast, in the method of the present invention, the reaction generally proceeds to completion in no more than about 6 hours. At this elevated temperature, it might have been expected that, given the high pH necessary for the reaction to proceed, the starting material, 6-bromo-6-deoxy-2,3-anhydro-D-manno-1,4-lactone, would be fragmented but surprisingly, it appears that this is not the case. It seems that the possibility of the starting material being lost is likely to have been the reason why the reaction has previously been carried out at 0° C.

In the process of the invention, the reaction proceeds to completion in not more than 6 hours, generally not more than 5 hours and more usually in not more than 4 hours, in the case of preparing L-gluconic acid. In contrast, the traditional process takes three days to proceed to completion. This reduction in time represents a considerable saving in the cost and the convenience of the process of the invention as compared to known processes. The inventors have found that the reaction temperature is important with a preferred reaction temperature being 45 to 55° C., and more preferably 45 to 50° C. The reaction may be conducted in an aqueous solvent, preferably a mixture of an organic solvent and water. Suitable organic solvents are polar solvents such as ketones, for example acetone or methylisobutyl ketone (MIBK).

The pH at which the reaction is conducted is also important with pH 12 being a minimum value. It is preferred, however, that the pH of the reaction mixture is at least pH 12.5, more preferably pH 13 and most preferably about pH 13.5-14.

The base used in the process of the invention is preferably an alkali or alkaline earth metal hydroxide, for example potassium, sodium or calcium hydroxide, although more favourable results are achieved using potassium and sodium hydroxide. The inventors have found that the best results are achieved using a molar ratio of hydroxide to 6-bromo-6-deoxy-2,3-anhydro-D-manno-1,4-lactone of between 1:2 and 1:4 but preferably 1:3. Using this amount of base ensures that the reaction mixture is sufficiently alkaline for the reaction to proceed.

The product of the reaction is a salt, the counter ion of which depends upon the base which is used in the process. However, if required, the free acid can be obtained by acidification of the product mixture, preferably with a strong acid such as hydrochloric acid, to a pH of about 1 to 2.5, or by ion exclusion chromatography. If the acid method is used, the product may be isolated from solution using conventional methods, for example by evaporation of the solvent.

It is also possible to obtain salts with alternative counter ions from the solution of the free acid by neutralising to pH 7 using an aqueous solution of a base having a suitable counter ion. For example, if a calcium salt is required, the acid solution can be treated with a base such as calcium carbonate or calcium acetate. The calcium gluconate salt is not particularly soluble and can be isolated by precipitation and filtration. Other more soluble salts, for example the sodium and potassium salts, can be obtained by neutralising the acidified solution as outlined above followed by recrystallisation of the required salt.

The process may include isolating the product, L-gluconic acid or a salt thereof, but for many applications, for instance if the product is to be used in another reaction, isolation is unnecessary and the product mixture from the process may be used without further purification.

L-gluconic acid or a salt thereof may, in turn be converted to L-glucose and therefore the process optionally further includes:

(ai) converting the L-gluconic acid or salt thereof to L-gluconolactone; and

(aii) converting the L-gluconolactone to L-glucose.

Steps (ai) and (aii) may be achieved by known methods. For example, a solution of an L-gluconic acid salt may be converted to the acid by acidification with a strong acid as described above. The solution may be heated to a temperature of about 40 to 60° C. and concentrated by removal of most of the solvent. Following this, an alcoholic solvent may be added to form L-gluconolactone.

L-gluconolactone may be converted to L-glucose by treatment with a reducing agent such as sodium borohydride. The reaction typically takes place at a temperature of −10 to 5° C. in an aqueous solvent and the product may be purified by ion exchange, followed by crystallisation, typically from water and/or an alcoholic solvent.

In a second aspect of the present invention there is provided a process for the preparation of epoxides (such as 6-bromo-6-deoxy-2,3-anhydro-D-manno-1,4-lactone) by reacting a bromohydrin (such as 2,6-dibromo-2,6-dideoxy-D-mannono-1,4-lactone) with a Lewis base in the presence of a catalytic amount of water.

Surprisingly, however the inventor has found that the reaction does not proceed particularly well under strictly anhydrous conditions and that improvements in the reaction time and the yield are obtained if a catalytic amount of water is present in the reaction mixture. The fact that the reaction actually proceeds more rapidly in the presence of a catalytic amount of water is an advantage as it means that it is not necessary to use expensive anhydrous reagents.

In general, the reaction is carried out in an organic solvent, typically a ketone such as acetone and methyl isobutyl ketone (MIBK). Other possible solvents include a non-polar solvent, for example hexane, benzene, toluene, diethyl ether, chloroform, ethyl acetate, and dichloromethane; a polar aprotic solvent, for example dioxane, tetrahydrofuran, acetone, methyl isopropyl ketone, methyl isobutyl ketone, butanone mesityl oxide, acetonitrile, dimethylformamide, and dimethylsulfoxide; and, less preferably, a polar protic solvent such as methanol, ethanol, n-propanol, isopropanol, n-butanol, formic acid and acetic acid. Preferred solvents include ketones, for example acetone, methyl isopropyl ketone, methyl isobutyl ketone, mesityl oxide, and butanone. Mixtures of two or more solvents are also contemplated.

The term “a catalytic amount of water” refers to the water content of the reaction solvent, which may be from about 0.05 to 2% by weight. However, it is preferred that the reaction solvent contains from about 0.2 to 0.8% or 0.9%, by weight, more preferably about 0.4 to 0.6% or 0.9%, by weight and typically about 0.5% by weight, or 0.75 to 0.8% by weight, for example 0.77% by weight, of water.

Any suitable Lewis base may be used but examples of particularly suitable bases include alkali metal fluorides and carbonates, for example potassium fluoride, potassium carbonate, caesium carbonate and rubidium fluoride. Potassium fluoride is particularly suitable as it is inexpensive and readily available. The inventors have found that the most favourable results are achieved using spray dried potassium fluoride as the Lewis base.

The reaction may be carried out on any suitable bromohydrin. Preferred bromohydrins include bromohydrins of aldonic acids and aldonolactones, and α-bromohydrin lactones. Particularly preferred bromohydrins include α-bromohydrin aldonolactones, for example allonolactone, altronolactone, galactonolactone, gluconolactone, gulonolactone, idonolactone, mannonolactone and talonolactone. The product of the reaction is preferably an epoxyaldonolactone, such as an α-epoxyaldonolactone.

The reaction is preferably carried out at a temperature of from 20 to 45° C., including room temperature, i.e. 20 to 25° C.; more preferably 30-45° C., even more preferably 30-40° C. Usually the reaction temperature is maintained at about 40° C. The reaction proceeds relatively rapidly and is usually complete in about 1 hour.

The process of the second aspect of the invention may be followed by conversion of the product in a subsequent reaction, for example, the conversion of 6-bromo-6-deoxy-2,3-anhydro-D-manno-1,4-lactone to L-gluconic acid, which may be achieved using the process of the first aspect of the invention.

In a third aspect of the present invention there is provided a process for the preparation of 2,6-dibromo-2,6-dideoxy-D-mannono-1,4-lactone:

(ci) reacting D-glucono-1,5-lactone or a salt thereof with a hydrogen halide at a temperature of from 40 to 60° C.;

(cii) adding methanol to the reaction mixture, adjusting the temperature of the reaction mixture to 40-55° C. and maintaining that temperature until the reaction has proceeded to completion.

Preferred hydrogen halides are hydrogen bromide, which may be used in a solvent such as acetic acid and hydrogen chloride, which may be in solution or in gaseous form.

The required temperature may be maintained by adjusting the reaction temperature to 40 to 50° C. after step (ci) and controlling the rate at which the methanol is added to the reaction mixture so as to ensure that the required temperature is achieved and maintained. After the addition of methanol is complete, the reaction temperature is maintained at 45 to 55° C. until the reaction is complete.

It is possible to determine whether the reaction is complete by monitoring at intervals. This may be done, for example, using thin layer chromatography at intervals in a manner known to those of skill in the art. The reaction is complete either when all of the starting material has disappeared or when the amount of starting material remains unchanged from one measurement to the next.

The temperature at which the reaction is carried out is important. If the reaction temperature is too low, the reaction will proceed at a rate which is unacceptably slow, whereas if it is too high, large amounts of a by-product formed in an elimination side reaction will be formed. A preferred temperature range for step (ci) of the reaction is from 50 to 60° C., with a range of 50 to 55° C., or 53 to 57° C., being more preferred and most preferably the temperature being maintained as near to 55° C. as possible. The reaction time for step (ci) is typically about 40 to 60 or 80 minutes, for example about 45 minutes, or 60 minutes.

In step (cii), some cooling is usually needed before the addition of the methanol, with the reaction temperature preferably being adjusted to about 25-35° C., for example about 30° C. Subsequently, the methanol is preferably added at a rate such that the temperature peaks at below 55° C. It has been found that addition of the methanol over a period of about 12 to 20 minutes is usually satisfactory if the reaction temperature is adjusted to about 30° C. before the addition of the methanol. In this case, the exotherm which occurs on the addition of methanol typically peaks at about 40 to 45° C. After the addition of the methanol, a preferred reaction temperature is 50 to 55° C. and generally, the reaction takes about 4 hours to proceed to completion after the methanol has been added.

Once the reaction is complete, additional steps may be used to extract and purify the product. A particularly effective optional method for the isolation of the product includes:

(ciii) distilling the product of step (cii);

(civ) dissolving the product of step (ciii) in MIBK and washing the solution with sodium hydrogen carbonate and water; and optionally

(cv) extracting the product by crystallisation or evaporation of the solvent.

Methyl isobutyl ketone (MIBK) is a particularly useful solvent for the isolation of the product as it dissolves the required product, 2,6-dibromo-2,6-dideoxy-D-mannono-1,4-lactone but not the more polar by-product.

If it is intended to use the product, 2,6-dibromo-2,6-dideoxy-D-mannono-1,4-lactone, for the synthesis of 6-bromo-6-deoxy-2,3-anhydro-D-manno-1,4-lactone, it is usually preferable to omit step (cv) and to use the solution obtained in step (civ) directly in the next step, particularly when the solvent used in step (civ) is methylisobutyl ketone. However, in this case, it is advantageous to wash the product of step (civ) with a weak base such as sodium bicarbonate so as to adjust the pH of the solution to 6 to 7 and adjust the water content of the solution to about 0.5 to 2%, more typically 0.7 to 1.5% and generally about 1% by weight.

Using the first, second and third processes described above, it is possible to convert D-glucono-1,5-lactone to L-gluconic acid.

### EXAMPLES

### Example 1

### Synthesis of 2,6-dibromo-2,6-dideoxy-D-mannono-1,4-lactone

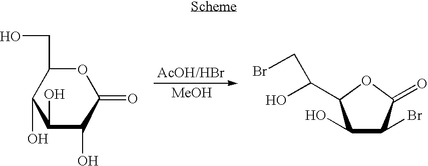

Procedure

D-Glucono-1,5-lactone (300 g) was charged into a 6 L jacketed reactor fitted with a mechanical overhead stirrer. Glacial HBr 33% (855 mL) was charged and the reaction was warmed to between 50-55° C. and held at 50-55° C. for 60 minutes. The solution was cooled to 30° C. then methanol (342 mL) was added over 13 minutes, the exotherm peaked at 42° C. The solution was warmed to 50-55° C. and was held at this temperature for 4 hours. Solvent was removed under reduced pressure with vessel jacket temperature set at 40° C., until the volume of product in the reactor was about 500 mL. MIBK (1,000 mL) was added and the solution was cooled to 0° C. The cold solution was washed with saturated aqueous sodium hydrogen carbonate (1,000 mL and 200 mL) followed by water (200 mL). MIBK was distilled under vacuum and the water content checked to ensure that it was below 1%. The solution can be used for the next stage.

Rf: 0.3 (toluene:acetone 4:1)

Mpt: 133-135° C.

¹H NMR δ (Cd₃CN): 4.94 (1H, d, J_{2,3}4.40 Hz, H-2), 4.57 (1H, dt, J_{3,2}4.40 Hz, J_{3,4}2.96 Hz, H-3), 4.42 (1H, dd, J_{4,3}2.96 Hz, J_{4,5}8.85 Hz, H-4), 4.15 (1H, m, H-5), 4.12 (1H, d, J5.80 Hz, H—OH), 3.55 (1H, dd, J_{gem} 11.08 Hz, J_{6,5}2.8 Hz, H-6), 3.5 (1H, d, 6.12 Hz, H—OH), 3.68 (1H, dd, J_{gem} 11.08 Hz, J_{6′,5} 5.16 Hz, H-6).

### Example 2

### Synthesis of 6-bromo-6-deoxy-2,3-anhydro-D-mannono-1,4-lactone

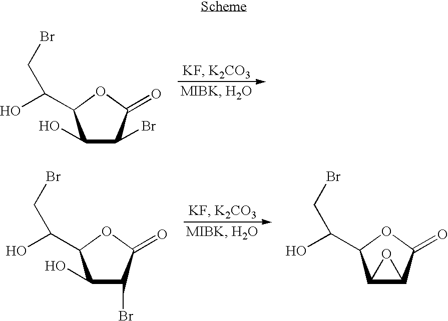

A solution of 2,6-dibromo-2,6-dideoxy-D-mannono-1,4-lactone (1,100 g) in MIBK (2,840 g) was charged into a 6 L jacketed vessel fitted with mechanical overhead stirring. The water content of the solution was adjusted to 0.77%. The solution was then warmed to 40° C. then potassium carbonate (1009 g, 2.2 molar equivalents) was added followed by potassium fluoride (636.5 g, 5 molar equivalents). The suspension was stirred for 1 hour at 40° C. by which time the reaction was complete. The suspension was filtered and the filter cake was washed with additional MIBK (4×400 mL). The solution containing of 6-bromo-6-deoxy-2,3-anhydro-D-mannono-1,4-lactone in MIBK was used for the next step without further purification.

### Example 3

### Synthesis of L-Gluconic Acid

The solution containing of 6-bromo-6-deoxy-2,3-anhydro-D-mannono-1,4-lactone in MIBK was charged into a 6 L jacketed vessel fitted with mechanical overhead stirrer. Water (1 mL to every 4 mL of MIBK solution) was added to the stirred solution followed by 3N sodium hydroxide solution until pH>13 was achieved. After 30 minutes the stirrer was stopped and the aqueous layer collected. The MIBK layer was washed with water (1 mL to every 4 mL of MIBK solution). The aqueous layers were combined and then heated to 45-50° C. for 4-5 hours by which time the reaction was complete. The pH is adjusted to 5-7 by the addition of aqueous HCl.

Formation and characterisation of the calcium salt is as follows:

A solution from the rearrangement reaction (which contained 2.9 g of epoxide) was acidified to pH 2 by addition of hydrochloric acid. To the acidified solution was added potassium carbonate until pH 7 was achieved. After 2 days, crystalline calcium-L-gluconate was isolated by filtration, washing the cold filter cake with cold aqueous methanol (7:3, 5 mL). The product was dried under vacuum to give an off white solid 1.42 g, 54% for the 2 steps.

[α]_{D}²² −5.5° (c=3, water)

¹H nmr δ (D₂O): 4.16 (1H, dd, J 1.2 Hz and J 3.4 Hz), 4.05 (1H), 3.79 (1H, dd), 3.76 (1H, m), 3.73 (1H, dd), 3.64 (dd, J 4.88 Hz and 11.6 Hz).

### Example 4

### Synthesis of L-Glucononlactone

Procedure

A stirred solution of crude potassium gluconate (0.24 moles) in water was acidified to pH 2.5 with concentrated HCl and then warmed to around 50° C. about 80% of the water was removed under vacuum distillation. To the warm solution isopropanol (800 mL) was added and the solution was heated to reflux azeotroping drying of the solution final volume about 200 mL. This lead to the formation of 1,4-lactone (major) and 1,5-lactone (minor). The solution was cooled to room temperature and neutralised by the addition of triethylamine to give pH 7. Inorganic salts were removed by filtration and the filtrate was collected and was used for the next step without further purification.

### Example 5

### Synthesis of L-Glucose

Procedure

The lactone solution (100 mL) containing about 0.14 moles was cooled to −5° C. to which ice cold water (100 mL) was added. To the solution was added sodium borohydride (5.1 g) in water 135 mL whilst maintaining the temperature below 5° C. The solution was stirred for 20 minutes and then quenched with acetic acid (2 mL). The solution was concentrated to about 100 mL and then purified by ion exchange chromatography passing down an acidic column (Dowex 50-X4™, 100 mL) and then a mild basic column (Dowex MWA-2™, 200 mL), fractions containing L-glucose were pooled and the product concentrated to a syrup. Product was crystallised from water, methanol and isopropanol to give crystalline L-glucose 9 g showing equal but opposite rotation to D-glucose with identical NMR spectrum.

### REFERENCES

1. E. Fischer, Ber. Dtsch. Chem. Ges. 23, (1890), 2611-2624.
2. C. Sowden, H. O. L. Fischer, J. Am. Chem. Soc., 69, (1947), 1963-1965.
3. R. Kuhn, P. Klesse, Chem. Ber., 91, (1958), 1989-1991.
4. V. Bilik, Chem. Zvesti, 26, (1972), 187-189.
5. W. Sowa, Can. J. Chem., 47, (1969), 3931-3934.
6. Hajko, A. Liptak, V. Pozsgay, Carbohydrate Res., 321, (1999), 116-120.
7. W. A. Szarek, G. W. Hay, D. M. Vyas, E. R. Ison, L. J. J. Hronowski, Can. J. Chem., 62, (1984), 671-674.
M. Shiozaki. J. Org. Chem., 56, (1991), 528-532.
8. S. Y. Ko, A. W. M. Lee, S. Masamune, L. A. Reed III, K. B. Sharpless, F. J. Walker, Tetrahedron, 46, (1990), 245-264.
9. M. Bednarski, S. Danishefsky, J. Am. Chem, Soc., 108, (1986), 7060-7067.
10. C. R. Johnson, A. Golebiowski, D. H. Steenma, J. Am. Chem. Soc., 114, (1992), 9414-9418.
11. I. Lundt, C. Pedersen, Synthesis, 7, 669-672, (1992).
12. Chemical Abstracts: 142135v, Vol. 75, (1971).
13. I. Lundt, R. Madsen, Top. Curr. Chem., 215, 177-191, (2001).

1. A process for preparing a compound, comprising:
reacting 2,6-dibromo-2,6-dideoxy-D-mannono-1,4-lactone with a Lewis base and a catalytic amount of water, to form 6-bromo-6-deoxy-2,3-anhydro-D-manno-1,4-lactone.

2. The process of claim 1, wherein the reacting is carried out in a solvent comprising a ketone.

3. The process of claim 1, wherein the catalytic amount of water is 0.05 to 2% by weight of a solvent in which the 6-bromo-6-deoxy-2,3-anhydro-D-manno-1,4-lactone is formed.

4. The process of claim 1, wherein the catalytic amount of water is 0.75 to 0.8% by weight of a solvent in which the 6-bromo-6-deoxy-2,3-anhydro-D-manno-1,4-lactone is formed.

5. The process of claim 1, wherein the Lewis base comprises at least one member selected from the group consisting of fluorides and carbonates.

6. The process of claim 1, wherein the Lewis base comprises potassium fluoride and potassium carbonate.

7. The process of claim 1, wherein the reacting is carried out at a temperature of 20 to 45° C.

8. The process of claim 2, wherein:
the catalytic amount of water is 0.05 to 2% by weight of the solvent,
the Lewis base comprises at least one member selected from the group consisting of fluorides and carbonates, and
the reacting is carried out at a temperature of 20 to 45° C.

9. The process of claim 1, further comprising forming L-glucose from the 6-bromo-6-deoxy-2,3-anhydro-D-manno-1,4-lactone.

10. The process of claim 8, further comprising forming L-glucose from the 6-bromo-6-deoxy-2,3-anhydro-D-manno-1,4-lactone.

11. A process for preparing a compound, comprising:
reacting a bromohydrin with a Lewis base and a catalytic amount of water, to form an epoxide.

12. The process of claim 11, wherein the bromohydrin is α-lactone.

13. The process of claim 11, wherein the bromohydrin is an α-bromohydrin lactone.

14. The process of claim 11, wherein the bromohydrin is an α-bromohydrin aldonolactone.

15. The process of claim 11, wherein the reacting is carried out in a solvent comprising a ketone.

16. The process of claim 11, wherein the catalytic amount of water is 0.05 to 2% by weight of a solvent in which the epoxide is formed.

17. The process of claim 11, wherein the Lewis base comprises at least one member selected from the group consisting of fluorides and carbonates.

18. The process of claim 11, wherein the reacting is carried out at a temperature of 20 to 45° C.

19-25. (canceled)

26. A process for preparing a compound, comprising:
reacting 6-bromo-6-deoxy-2,3-anhydro-D-manno-1,4-lactone with a base at a pH of at least 12 and at a temperature of 45 to 55° C., to form L-gluconic acid or a salt thereof.

27-30. (canceled)

31. A process for preparing a compound, comprising:
reacting D-glucono-1,5-lactone or a salt thereof with a hydrogen halide at a temperature of from 40 to 60° C., to form a reaction mixture; and
adding methanol to the reaction mixture, to form 2,6-dibromo-2,6-dideoxy-D-mannono-1,4-lactone.

32-37. (canceled)
